(19) United States
(12) Patent Application Publication
(10) Pub. No.: US 2002/0103261 A1
(43) Pub. Date: Aug. 1, 2002

(54) Compositions for injection or intravenous administration for the treatment of internal infection or inflammation in humans and animals

(76) Inventor: Dusan Ninkov, Orange City, IA (US)

Correspondence Address: MERCHANT & GOULD PC, P.O. BOX 2903, MINNEAPOLIS, MN, 55402-0903, US

(21) Appl. No.: 09/974,719

(22) Filed: Oct. 9, 2001

Related U.S. Application Data
(60) Provisional application No. 60/238,501, filed on Oct. 6, 2000.
(60) Provisional application No. 60/247,157, filed on Nov. 10, 2000.
(60) Provisional application No. 60/277,121, filed on Mar. 19, 2001.
(60) Provisional application No. 60/288,531, filed on May 3, 2001.

(51) Int. Cl. A61K 31/05

(52) U.S. Cl. 514/731

(57) ABSTRACT

A pharmaceutical compositions is provided that include oil extract from plants from the Labiatae family. The compositions can be formulated by combining extracts of essential oils from plants of the Labiatae family with a Group I base. The pharmaceutical compositions are formulated for subcutaneous, intradermal, intramuscular or intravenous injection of mammals, including humans.

This application claims priority to U.S. Provisional Application No. 60/238,501 filed Oct. 6, 2000; No. 60/247,157 filed Nov. 1, 2000; No. 60/277,121 filed Mar. 19, 2001; and No. 60/288,531 filed May 3, 2001.

### FIELD OF THE INVENTION

The invention generally relates to pharmaceutical compositions for injection or intravenous administration which include oil extract from plants from the Labiatae and Verbenacea family.

### BACKGROUND OF THE INVENTION

The common name for members of the Labiatae, a large family of chiefly annual or perennial herbs, is the “mint family.” The mint family is classified in the division Magnoliphyta, class Magnoliopsida, order Lamiales. The mint family includes about 200 genera, such as Salvia (sage), Rosmarinus (rosemary), Mentha (mint), Ocimum (basil), Thymus (thyme), Marrubium (hoarhound), Monarda (horse-mint), Trichostema (bluecurls), Teucrium, Hyptis, Physostegia, Lamium (henbit), Stachys, Scutellaria (skullcap), Nepeta (catmint). Members of the Verbenaceae family include Lippia (Mexican Oregano) and Lycopus.

The plants in the mint family are typically shrubby or climbing, although some exist as small trees. The plants are found throughout the world.

The mint family is well known for the aromatic volatile or essential oils in the foliage, which are used in perfumes, flavorings, and medicines. Among the more important essential oils are those derived from sage, lavender, rosemary, patchouli, and the true mints. Many of the commonly used potherbs are from the mint family, e.g., basil, thyme, savory, marjoram, oregano, as well as those plants previously mentioned.

Many of these plants such as catnip, pennyroyal, hyssop, self-heal, the horehound of confectionery have a history of medicinal use in domestic remedies. Others are used as curative teas, for example, bee balm and yerba buena.

The true mints belong to the genus Mentha. Catnip or catmint refers to a strong-scented perennial herb (Nepeta cataria) of the family Labiatae. Catnip is native to Europe and Asia and naturalized in the United States. Although best known for its stimulating effect on cats, tea of the leaves and tops of the catnip plant have long been used as a domestic remedy for various ailments. For example, dry leaves from Nepeta cataria have been used for the production of tea, to treat restlessness, nervousness, insanity, and as a tonic for colic and carminative.

U.S. Pat. No. 5,990,178 discloses pharmaceutical compositions for treating a disease in poultry induced by hemoflagellates. The pharmaceutical compositions disclosed therein contain thymol (5-methyl-2[1-methylethyl]phenol) and/or carvacrol (5-isopropyl-2-methylphenol). Thymol (also referred to as isopropyl-cresol) and carvacrol (also referred to as isopropyl-o-cresol) can be synthetic or obtained from oil extract from plants such as Origanum vulgaris, Thymus vulgaris, Mentha piperita, Thymus sepilum, Saturia hortensis, Saturea montana, Saturea subricata, Carum corticum, Thymus zugus, Ocimum gratisimum, Moranda pungata, Moslajananoica, and Salva officinalis.

WO 96/37210 discloses pharmaceutical compounds which contain etheric oils from plants including Origanum vulgaris, Thymus vulgaris, Mentha piperita, Thymus serpilum, Saturea horensis, Saturea montana, Saturea subricata, Carum cortiucm, Thymus zugis, Ocimum gratisimum, Moranda pungtata, Mosla japanoica and Salva officinalis.

Commonly assigned co-pending U.S. patent application Ser. No. 09/499,197, the disclosure of which is incorporated by reference herein in its entirety, discloses similar compounds.

### SUMMARY OF THE INVENTION

This disclosure provides pharmaceutical compositions which include oil extract from plants from the Labiatae and Verbenacea family. In particular, the compositions can be formulated by combining extracts of an essential oil with a Group I salt. It is believed that the antimicrobial activity of the pharmaceutical composition is due to the presence of organic phenolic compounds, such as isopropyl-o-cresol, (5-isopropyl-2-methylphenol) and/or isopropyl-cresol (5-methyl-2[1-methylethyl]phenol) in the oil extract from the plants.

Suitable plants from the Labiatae and Verbenacea family include, but are not limited to, Ocimum spp., Saturea spp., Monarda spp, Origanum spp, Thymus spp., Mentha spp., Nepeta spp., Teucrium gnaphalodes, Teucrium polium, Teucrim divaricatum, Teucrim kotschyanum, Micromeria myrifolia, Calamintha nepeta, Rosmarinus officinalis, Myrtus communis, Acinos suaveolens, Dictamnus albus, Micromeriafruticosa, Cunila origanoides, Mosla Japonoica Maxymowitz, Pycnanthemum nudum, Micromeria Juliana, Piper betel, Trachyspermum ammi and Lippia graveolens. In a preferred composition, the plant is Nepeta racemosa or Nepeta Cataria.

Suitable salts include Group I bases formed from a Group I cation. Preferred bases include Group I hydroxide bases and the most preferred bases are sodium hydroxide and potassium hydroxide.

The pharmaceutical compositions of this invention are formulated to be injected subcutaneously, intradermally or intramuscularly; or to be administrated intravenously. The pharmaceutical compositions of this invention are meant to treat internal infections and inflammations of humans and animals.

### BRIEF DESCRIPTION OF THE DRAWINGS

FIG. 1 shows a structural formula for isopropyl-o-cresol or 2-methyl-5 [1-methylethyl]phenol)

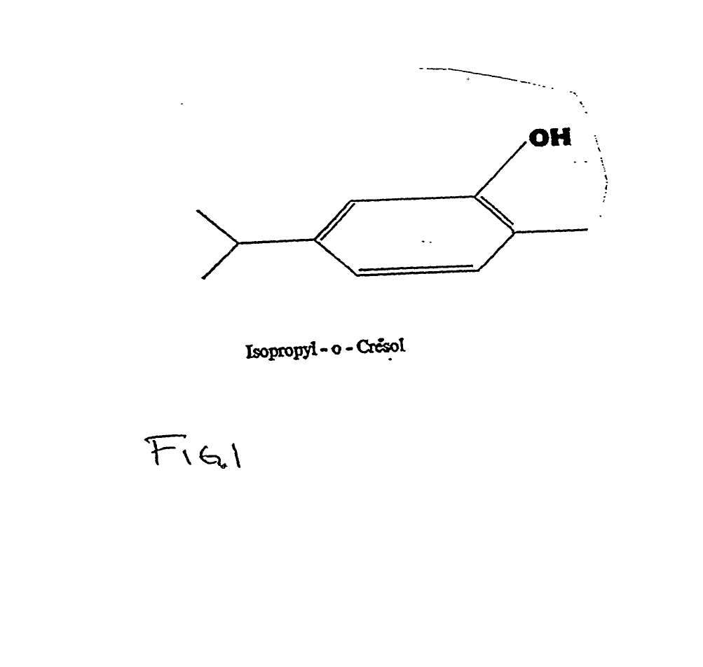

FIG. 2 shows a structural formula for sodium isopropyl-o-cresol.

FIG. 3 shows a structural formula for potassium isopropyl-o-cresol.

FIG. 4 shows a structured formula for isopropyl-cresol (5-methyl-2[1-methylethyl]phenol)

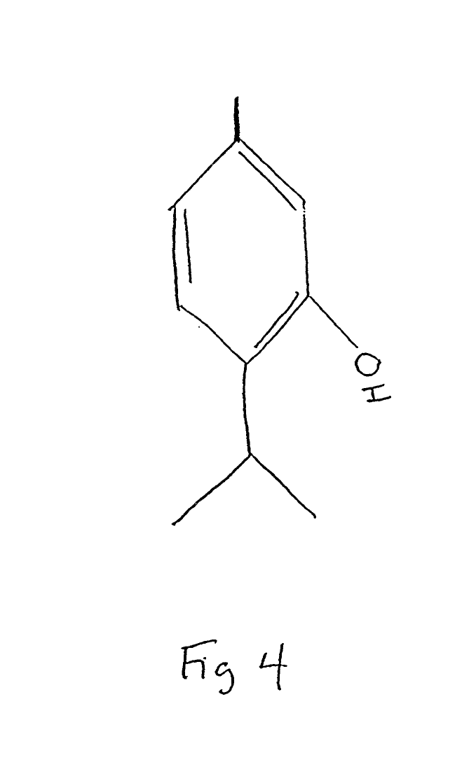

FIG. 5 shows a structural formula for sodium isopropyl-cresol.

FIG. 6 shows a structural formula for potassium isopropyl-cresol.

FIG. 7 is a schematic showing the chemical reactions between isopropyl-0-cresol and isopropyl-cresol and sodium and potassium hydroxide.

### DETAILED DESCRIPTION OF THE INVENTION

The invention provides pharmaceutical compositions that include an oil extract from plants from the Labiatae and Verbenaceae family. In particular, the antimicrobial pharmaceutical compositions include an organic phenolic compound such as isopropyl-o-cresol (5-isopropyl-2-methylphenol) and/or isopropyl-cresol (5-methyl-2[1-methylethyl]phenol). The organic phenolic compound can be obtained from plant oil extracts or synthesized by known methods. In one embodiment, the organic phenolic compound is combined with a Group I base to form a base reacted compound. Both the unreacted organic phenolic compound and the base reacted organic phenolic compounds are referred to herein as antimicrobial compounds.

The pharmaceutical compositions are suitable for treating internal microbial infections and internal inflammation processes in animals, including, humans and livestock, including but not limited to horses, cows, pigs, sheep, goats, rabbits, dogs, cats and poultry, including, but not limited to chickens, turkeys, ducks and pet birds.

Because the antimicrobial compounds are degraded by enzymes, the pharmaceutical compositions are particularly well suited for treating microbial infections in livestock. Little residue from the antimicrobial compound is found in products from treated livestock, such as milk, eggs, and meat. Organic phenolic compounds such as isopropyl-o-cresol and isopropyl-cresol are degraded by enzymes into inactive metabolites. The metabolites can be excreted in the urine (approx. 90%) or expired from the lungs (10%) in the form of CO₂. Additional information on the degradation of isopropyl-o-cresol and isopropyl-cresol, can be found in US Pharmacopoeia, British and European Pharmacopoeia, and Textbook of Veterinary Physiology, by Prof. Dr. James G. Cunningham, Ph.D., 2nd edition. The text of all three references is hereby incorporated by reference herein.

Additionally, antimicrobial compounds of the invention do not appear to be mutagenic or carcinogenic.

Furthermore, it is believed that the efficacy of the antimicrobial compounds will not be compromised because of pathogen resistance. It is believed that the activity of the antimicrobial compounds are similar to the activity of benzyl alcohol, phenol and polyphenols in that the antimicrobial compounds destroy the cell membranes of the microorganism to cause cell death. The British Pharmacopoeia, Edition 1996 reports that microorganisms do not build resistance to benzyl alcohol, phenols, polyphenols, and similar products.

As used herein, the term “antimicrobial activity” includes bactericidal, fungicidal, protozoanicidal, and other disinfective activity.

I. Antimicrobial Compound

A. Organic Phenolic Compound

The antimicrobial compounds of the invention include an organic phenolic compound such as isopropyl-o-cresol (5-isopropyl-2-methylphenol) or isopropyl-cresol (5-methyl-2[1-methylethyl]phenol). In one embodiment, the antimicrobial compound is an organic phenolic compound combined with a Group I base.

Isopropyl-o-cresol is a crystal with a boiling point of about 233° C. at atmospheric pressure. Isopropyl-cresol is a liquid that has a boiling point at atmospheric pressure of 237-238° C. Both compounds volatilize in water vapor.

Organic phenolic compounds can be made synthetically by known methods, or can be obtained from plant oil extract. Preferably, the oil is extracted from a member of the Labiatae or Verbenaceae family. The Labia tae family includes about 200 genera, such as Salvia, Rosmarinus, Mentha, Ocimum, Thymus, Marrubium, Monarda, Trichostema, Teucrium, Hyptis, Physostegia, Lamium, Stachys, Scutellaria and Lycopus. Suitable plants include, but are not limited to, Ocimum spp., Saturea spp., Monarda spp, Origanum spp, Thymus spp., Men tha spp., Nepeta spp., Teucrium gnaphalodes, Teucrium polium, Teucrim divaricatum, Teucrim kotschyanum, Micromeria myrifolia, Calamintha nepeta, Rosmarinus officinalis, Myrtus communis, Acinos suaveolens, Dictamnus albus, Micromeria fruticosa, Cunila origanoides, Mosla Japonoica Maxymowitz, Pycnanthemum nudum, Micromeria Juliana, Piper betel, Trachyspermum ammi, Lippia graveolens as well as others. In a preferred composition, the oil extract is from plant of the species Nepeta including, but not limited to Nepeta racemosa (catmint), Nepeta citriodora, Nepeta elliptica, Nepeta hindostoma, Nepeta lanceolata, Nepeta leucophylla, Nepeta longiobracteata, Nepeta mussinii, Nepeta nepetella, Nepeta sibthorpii, Nepeta subsessilis and Nepeta tuberosa.

Organic phenolic compounds such as isopropyl-o-cresol and isopropyl-cresol are soluble in lipids. It is believed that the antimicrobial activity of the organic phenolic compounds is due to the destruction of lipids in the microorganism's cell membrane.

1. Synthetic Production of Organic Phenolic Compound

Methods for synthetically producing organic phenolic compounds such as isopropyl-o-cresol and isopropyl-cresol are known. See, for example, Organic Chemistry by Morrison & Boyd 2d ed. 1971 at page 815. Additionally, these compounds are available from chemical manufacturers and are listed in the Merck Index. However, it is generally preferred that the organic phenolic compound be extracted from plants instead of being chemically synthesized. Because phenol is used to synthesize isopropyl-o-cresol and isopropyl-cresol, the resulting synthetic product tends to contain residual phenol (less than 1%). It may be undesirable to administer a composition containing phenol to an animal because phenol can be mutagenic and carcinogenic.

2. Extraction of Isopropyl-o-cresol from Plants

i. Cultivating the Plant

Plants of the Labiatae and Verbenacea families are found throughout the world and are relatively easy to cultivate. To cultivate the plants, seeds, preferably those with a high percentage (e.g., at least about 70 wt %, more preferably at least about 80 wt %, of organic phenolic compound), are planted in fine loose soil, preferably in a sub-tropical climate. Hybrid seeds having a high percentage of organic phenolic compounds can be produced by known techniques. The seeds are then cultivated using known agricultural techniques, such as watering, and artificial fertilizing.

Because the leaves contain a high amount of oil upon blossoming, it is preferred that the plants be harvested soon after the plants begin to blossom. Preferably, the plants are harvested within 24 hours after blossoming, more preferably within 12 hours after blossoming. Most preferably, harvesting is undertaken early in the morning or late in the evening hours when the leaves are not exposed to the sun.

Because the majority of the oil is found in the leaves and blossoms of the plant, it is preferred that the leaves and blossoms be utilized in the extraction process. Use of other parts of the plant may increase impurities and decrease yield.

ii. Extracting Oil from the Plant

Oil containing organic phenolic compounds can be extracted from either dried or fresh plants, or both. If the plant is dried, the drying process is preferably undertaken in special drying houses that are constructed to allow constant, free circulation of air. Preferably, the harvested leaves and blossoms should not be exposed to direct sunlight, as exposure to sunlight may reduce the amount of active material present in the leaves.

To dry the product, the leaves and blossoms are arranged in layers of 20-25 cm thick. To promote uniform drying, the layers should be turned up-side-down either manually or mechanically daily, preferably more than once a day, more preferably multiple times a day, such as four times a day, preferably during the first few days of drying, typically within the first three days. Generally, the leaves are dried for about 7 to 8 days.

After the leaves and blossoms are dried, the oil can be extracted by known methods, including distillation, for example, steam distillation. Preferably, the oil is extracted in a two stage distillation process (double distillation). Preferably, the oil is first extracted by steam distillation (at a temperature of about 100° C.) to remove most impurities. Typically, after the first steam distillation, the extracted oil contains about 3% to about 4% by weight isopropyl-cresol; about 60% to about 70% isopropyl-o-cresol and about 26% to about 37% by weight impurities.

The oil is then re-distilled at a temperature between about 180° C. to about 200° C. to remove additional impurities. Preferably, the redistillation is performed twice (double re-distillation). If a double re-distillation process is used, the oil typically has a purity of greater than 90%, more preferably greater than 95%, and even up to 99%. Although yield tends to be lower when a double distillation process is used, typically about 1 to 10 kilograms, more typically about 3 to 7 kilograms of oil, are obtained for every 100 kilograms of dried leaves and blossoms.

In a steam distillation process, the distillation column generally has two output tubes: one for oil (at the base of the column) and one for water vapor (at the top of the column). A water source is positioned under the leaves and blossoms and is heated to about 100° C. preferably under a pressure of about 20 bar to about 25 bar (increased pressure will tend to reduce the distillation time). The steam passes through the leaves and blossoms, thereby creating oil droplets. Because the water vapor is lighter than the oil droplets, the water droplets flow out of the output tube positioned at the top of the distillation column and the oil droplets flow out of the output tube positioned at the base of the distillation column. The distillation process is carried out for about 1 to about 5 hours, more typically about 2 to about 3 hours.

The antimicrobial compounds of the invention are then purified further using chromatographic techniques.

B. Group I Salt

In one embodiment, the organic phenolic compound is combined with a salt, preferably a Group I salt. A Group I salt refers to an ionic molecule that has as its cation one of the elements in Group I of the periodic chart of elements. Group I salts as used herein include any Group I cation in combination with any anion. Examples of Group I salts include for example Group I chlorides and Group I bases such as Group I hydroxides. Preferred Group I salts include Group I bases, more preferrably Group I hydroxide bases, most preferably the Group I base is sodium hydroxide and/or potassium hydroxide. The Group I hydroxide is combined with the organic phenolic compound to form a base reacted antimicrobial compound comprising the deprotonated organic phenolic compound associated with the Group I cation. Specific methods of forming these compounds of the invention are provided below.

The Group I bases are combined with the organic phenolic compounds in ratios (by weight) in the range of about 50 wt % to about 75 wt % organic phenolic compound to about 25 wt % to about 50 wt % Group I base. Preferably, the ratio of organic phenolic compound to Group I base is within the range of about 55 wt % to about 70 wt % organic phenolic compound to about 30 wt % to about 45 wt % Group I base More preferably, the ratio of organic phenolic compound to Group I base is about 55 wt % to about 60wt % organic phenolic compound to about 40 wt % to about 45 wt % Group I base.

It is believed that the sodium and potassium ions, along with the deprotonated organic phenolic compound readily pass through or destroy the cell membrane. The association of the organic phenolic compound with sodium or potassium appears to increase the rate of pathogen destruction.

C. Reaction to form Antimicrobial Compound

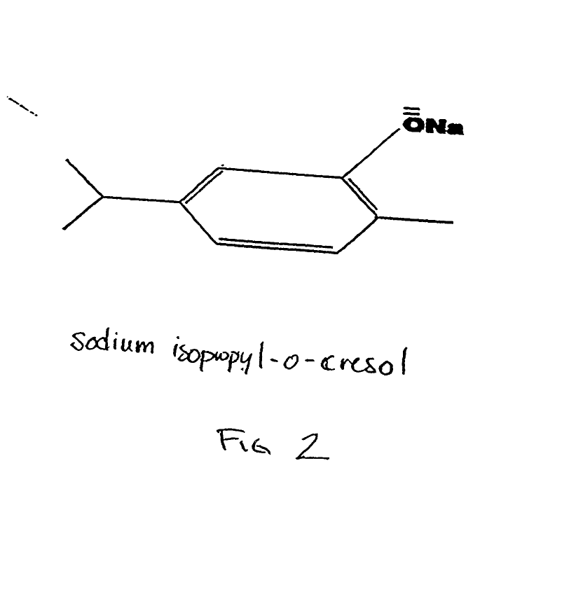

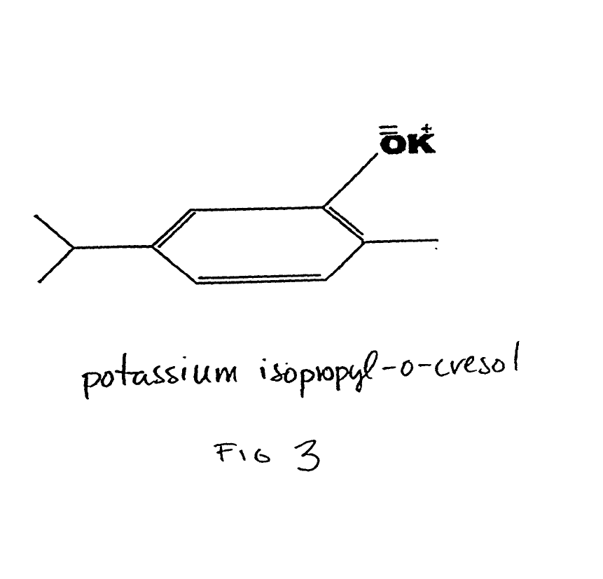

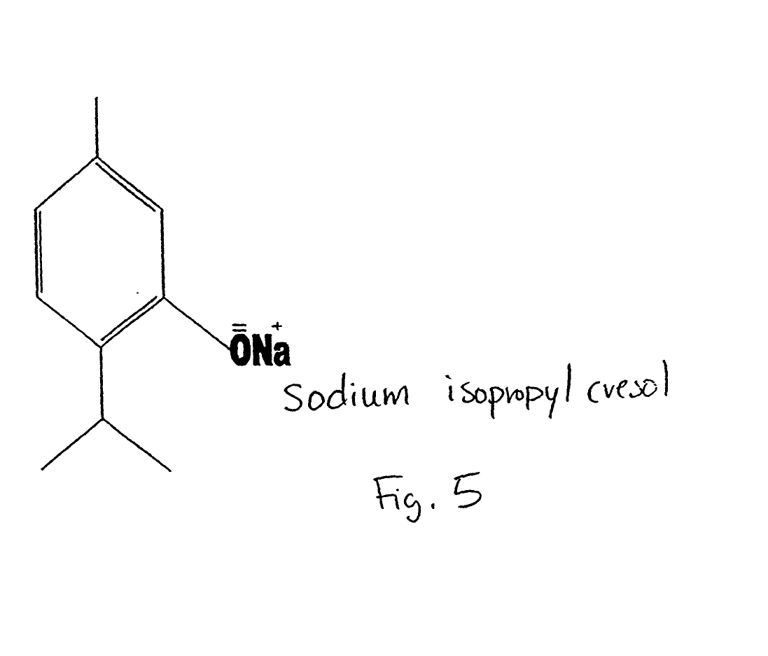

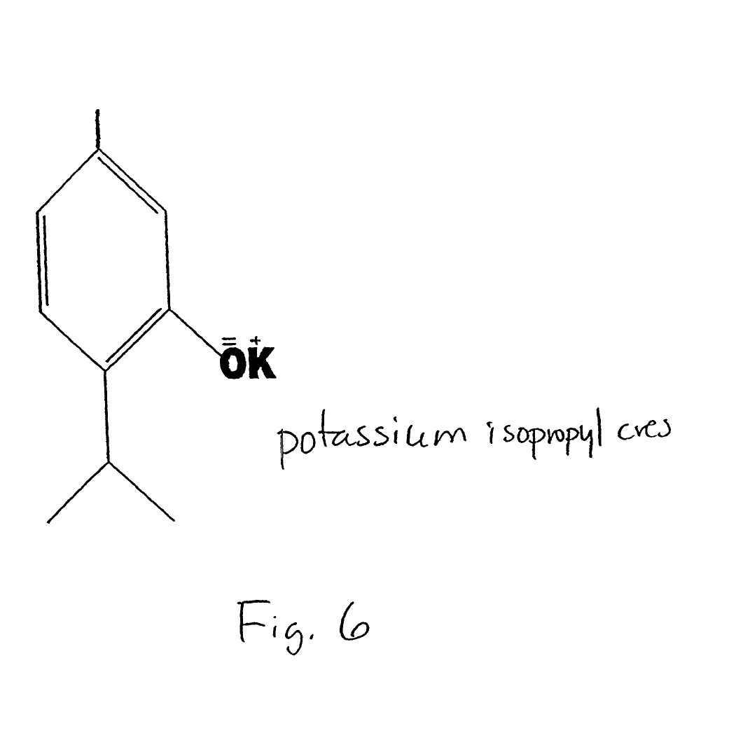

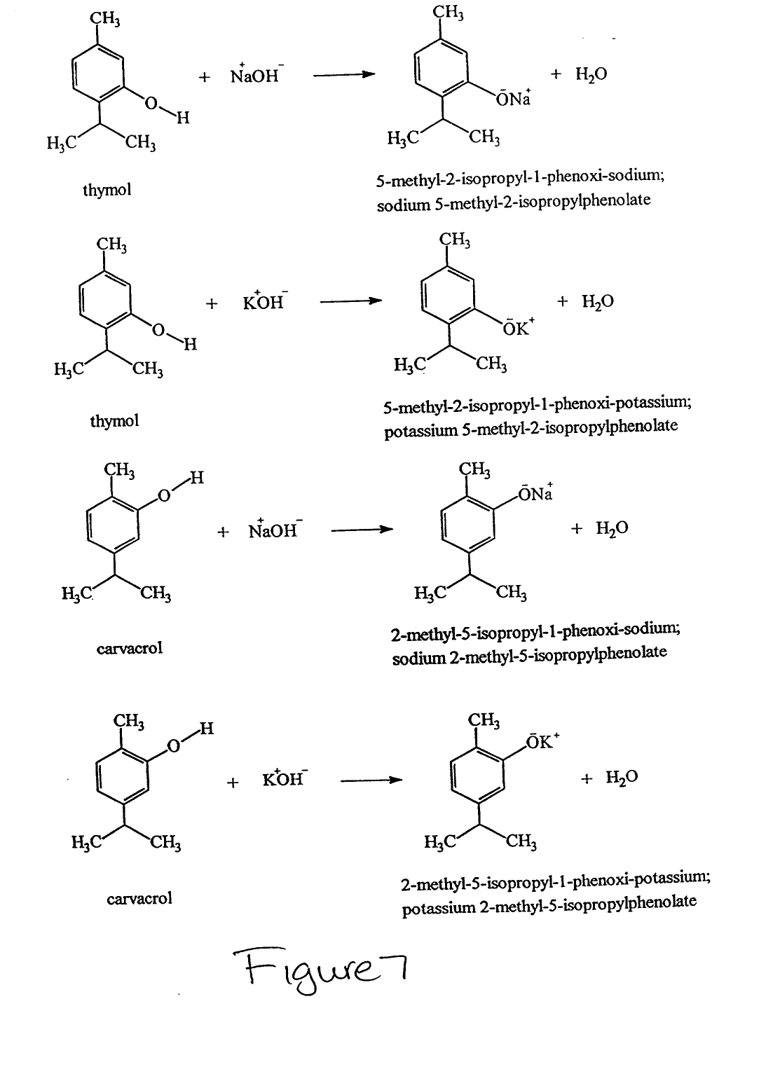

As used herein, the term “antimicrobial compound” refers to both unreacted organic phenolic compounds and compounds formed by reacting an organic phenolic compound extracted from a plant of the Labiatae and/or Verbenacae family with a salt. In some instances, the antimicrobial compound formed by reacting an organic phenolic compound with a base may be referred to as a “base reacted” compound. The antimicrobial compound may also be referred to as the “active ingredient.” An “antimicrobial compound” may refer to a compound formed by chemically reacting isopropyl-o-cresol or isopropyl-cresol with sodium hydroxide (See, FIGS. 2 and 5) or with potassium hydroxide (See, FIGS. 3 and 6). FIG. 7 shows the chemical reaction of thymol and carvacrol with sodium and potassium hydroxide.

An “antimicrobial compound” may also refer to a compound formed by chemically reacting isopropyl-o-cresol or isopropyl-cresol with an organic acid or a Group I salt as specifically disclosed in commonly assigned co-pending U.S. patent application Ser. No. 09/499,197; the specifically referred to disclosure of which is incorporated herein in its entirety by reference.

As used herein, the term “reacting” refers to a process in which the organic phenolic compound is chemically modified (as compared to the formation of a solution). In the formation of an antimicrobial compound with a Group I base, the reaction of the organic phenolic compound involves the deprotonation of the alcohol moiety to form an aryl oxide anion which then associates with the Group I cation in solution.

Sodium isopropyl-o-cresol is a light brown solid that dissolves in water to form a yellowish-brown solution. Potassium isopropyl-o-cresol is a gray solid that dissolves in water to form a grayish solution. Sodium isopropyl-cresol is a brown solid that dissolves in water to form a brownish solution. Potassium isopropyl-cresol is a dark brown solid that dissolves in water to form a brown solution.

1. Synthesis

For example, a base reacted isopropyl-o-cresol can be formed by reacting isopropyl-o-cresol with XOH (X=Na or K). Preferably XOH is pulverized through use of for example, a mortar and pestle. The XOH is also preferably dissolved in ethanol. Preferably, the ethanolic solution of XOH is heated to facilitate the dissolution of the XOH into X⁺ and OH⁻. Preferably, the ethanolic solution of XOH is prepared in a conical flask to reduce absorption of CO₂ and prevent the formation of Na₂CO₃ or K₂CO₃.

The concentration of the XOH ethanolic solution generally ranges from about 0.25 M to 1.0 M. Preferably from about 0.50 M to 0.75 M. Most preferably from about 0.60 M to 0.70 M.

For example, an ethanolic solution of NaOH can be formed by combining 0.8 g (20 mmol) pulverized NaOH with 30 ml anhydrous ethanol. The NaOH can be pulverized using a mortar and pestle. An ethanolic solution of KOH can be formed by combining 1.1 g (20 mmol) pulverized KOH with 30 ml anhydrous ethanol. The KOH can be pulverized using a mortar and pestle. Both of these preparations produce a solution that has a concentration of about 0.66 M NaOH or KOH respectively.

The organic phenolic compound (thymol and/or carvacrol) is then added to the ethanolic solution and mixed at about 180 RPM for from about 3 to 10 minutes, preferably up to about five (5) minutes. Once the solution is evaporated, the ethanol is evaporated to obtain a base reacted antimicrobial compound in a solid form. The base reacted antimicrobial compound can be dried at about 60° C.-70° C., in a vacuum for about 6-7 hours to remove water that may have been produced by the chemical reaction.

Alternately, a sodium methoxide can be reacted with an organic phenolic compound to form a base reacted antimicrobial compound. In this embodiment, the product of the chemical reaction is ethanol, instead of water.

2. Purification

If desired, the solid base-reacted antimicrobial compound can be purified, for example, by recrystallization. In purification by recrystallization, a solvent is selected in which the compound is soluble at higher temperatures, but only slightly soluble at lower temperatures, so that the compound will pass from solution to precipitate at a lower temperature while impurities remain in solution.

For example, the antimicrobial compound can be combined with ethanol to produce a suspension. The suspension is then heated until it boils (between 60° C.-70° C.). Ethanol is added dropwise to the heated suspension until the base-reacted antimicrobial compound is completely dissolved. The mixture is then cooled to precipitate the purified compound. Generally, pure compound will precipitate at a lower temperature than impurities.

The antimicrobial compound can also be purified by certain chromatographic methods, including but not limited to solid-liquid, liquid-liquid, and gas-liquid type chromatography. Examples of solid-liquid type chromatographic methods that could be utilized include column chromatography, gel chromatography, dry-column chromatography, or high performance liquid chromatography (HPLC).

In one embodiment, the organic phenolic compounds are combined. For example, one or more organic phenolic compounds that have been reacted with a Group I salt can be combined with one or more organic phenolic compounds that have not been reacted with a Group I salt. In another embodiment, one or more organic phenolic compounds that have been reacted with a Group I base can be combined with one or more organic phenolic compounds that have been reacted with a different Group I base. For example, an antimicrobial compound obtained by reacting sodium hydroxide with isopropyl-o-cresol can be mixed with an antimicrobial compound obtained by reacting potassium hydroxide with isopropyl-o-cresol. Additionally, an antimicrobial compound obtained by reacting sodium hydroxide with isopropyl-cresol can further be mixed with an antimicrobial compound obtained by reacting potassium hydroxide with isopropyl-cresol.

In one embodiment, an antimicrobial compound formed by reacting an organic phenolic compound with a sodium cation is combined with an antimicrobial compound formed by reacting an organic phenolic compound with a potassium cation. More preferably, the sodium and potassium reacted antimicrobial compounds are combined in approximately equal amounts (i.e., within about 10 wt % of each other, more preferably within about 5 wt % of each other, most preferably within about 1 wt % of each other). The sodium and potassium base reacted antimicrobial compounds are typically mixed at 150 revolutions per minute for 5 minutes to produce a homogenous mixture.

In yet another embodiment, base reacted isopropyl-o-cresol and base reacted isopropyl-cresol are combined. Preferably a mixture containing sodium and potassium base reacted isopropyl-o-cresol is combined with a mixture containing sodium and potassium base reacted isopropyl-cresol. More preferably, the mixture contains more isopropyl-o-cresol antimicrobial compound than isopropyl-cresol antimicrobial compound. For example, the mixture can contain between about 1 wt % to about 45 wt % isopropyl cresol antimicrobial compound and between about 55 wt % and about 99 wt % isopropyl-o-cresol antimicrobial compound; more preferably between about 1 wt % to about 25 wt % isopropyl cresol antimicrobial compound and between about 75 wt % and about 99 wt % isopropyl-o-cresol antimicrobial compound; more preferably between about 1 wt % to about 10 wt % isopropyl cresol antimicrobial compound and between about 90 wt % and about 99 wt % isopropyl-o-cresol antimicrobial compound; most preferably between about 1 wt % to about 5 wt % isopropyl cresol antimicrobial compound and between about 95 wt % and about 99 wt % isopropyl-o-cresol antimicrobial compound. Again, mixing is typically carried out at 150 revolutions per minute for at least 5 minutes to produce a homogenous mixture.

II. Pharmaceutical Compositions

The antimicrobial compound can be used alone, or as part of a pharmaceutical composition. As used herein, the term “pharmaceutical composition” refers to a composition which includes at least one antimicrobial compound and a pharmaceutically acceptable carrier. The term “pharmaceutical composition” can refer to a combination of unmodified organic phenolic compound and/or base reacted organic phenolic compound a pharmaceutically acceptable carrier. This definition of “pharmaceutical composition” includes essential oils obtained from plants as well as synthetically produced organic phenolic compounds combined with acceptable carriers.

The methods of treatment of this invention include administration through parenteral preparations. Parenteral preparations are introduced directly into the body fluid systems composing the intra- or extra-cellular fluid compartments, the lymphatic system, or the blood circulatory system. Since the protective characteristics of the skin and mucous membranes are circumvented by parenteral administration, the introduction of toxic agents and microorganisms is of great concern.

Parenteral preparations can be classified into five general categories: (1) solutions ready to be injected; (2) dry products that are to be solubilized just prior to injection; (3) suspensions ready for injection; (4) dry, insoluble products ready to be combined with a carrier just prior to use, and (5) emulsions. Parenteral preparations can be administered by one or more routes, such as intravenous, subcutaneous, intradermal, intramuscular, intraspinal, intracisternal, and intrathecal. The nature and purpose of the preparation will determine the ultimate route of delivery. The specific delivery route that is chosen will place further constraints on the formulation. One advantage of parenteral administration is that it avoids inactivation by digestive processes and irregularities due to intestinal absorption.

Typically, the preparation of a parenteral formulation of a pharmaceutical begins with the selection of the carriers to be used. The carrier typically has no therapeutic activity. Absorption of the pharmaceutical from the carrier can be affected by the viscosity of the carrier, its capacity for wetting the solid particles, the solubility equilibrium produced by the carrier, and the distribution coefficient between the carrier and the aqueous system of the body.

Acceptable carriers for parenteral preparations include distilled water; aqueous carriers such as sodium chloride injection, ringer's injection, dextrose injection, dextrose and sodium chloride injection, and lactated ringer's injection; water-miscible carriers such as ethyl alcohol, polyethylene glycol and propylene glycol; and nonaqueous carriers such as fixed oils. Preferably the fixed oil is of vegetable origin because such fixed oils tend to be metabolized, are a liquid at room temperature, and do not become rancid rapidly. The oils most commonly used are corn oil, cottonseed oil, peanut oil, and sesame oil. However, any vegetable oil that fits the above parameters may be used.

Preferably, the carrier utilized in a parenteral preparation that will be injected subcutaneously, intradermally or intramuscularly is a nonaqueous carrier. More preferably, the carrier for such parenteral preparations is a highly purified olive oil.

Preferably, the carrier utilized in a parenteral preparation that will be injected intravenously is either water or an aqueous carrier. More preferably, the carrier for such parenteral preparations is a sodium chloride solution.

Preferably, the pharmaceutical composition is in unit dosage form. In such form, the preparation is subdivided into unit doses containing appropriate quantities of the antimicrobial compound. The unit dosage form can be a packaged preparation, the package containing discrete quantities of preparation, for example, packaged injection amounts.

The quantity of antimicrobial compound in a unit dose may be varied or adjusted from 1 mg to 1000 mg according to the particular application. The antimicrobial compounds are typically administered at an initial dosage of about 5 mg to about 50 mg per kilogram daily. The dosages, however, may be varied depending upon the requirements of the animal being treated, the severity of the condition being treated and the compound employed. Determination of the proper dosage for a particular situation is within the skill of the art. For convenience, the total daily dosage may be divided and administered in portions during the day if desired.

A. Combinations

A pharmaceutical composition may include one or more organic phenolic compounds (e.g., isopropyl-o-cresol and/or isopropyl cresol), one or more base reacted antimicrobial compounds (e.g., sodium isopropyl-o-cresol; potassium isopropyl-o-cresol; sodium isopropyl-cresol; and/or potassium isopropyl-cresol), or combinations thereof. In one embodiment, the pharmaceutical composition includes both a sodium and a potassium salt of an organic phenolic compound.

Most preferably, the pharmaceutical composition includes antimicrobial compounds formulated as both sodium and potassium salts of the organic phenolic compound. Preferably, the total amount of sodium and potassium organic phenolic salts make up approximately 0.1 wt % to about 15 wt %, more preferably about 0.5 wt % to about 10 wt %, most preferably about 0.5 wt % to about 0.8 wt % of the pharmaceutical composition for an intravenous composition and 3.5 wt % to about 10 wt % for an injectable composition. Preferably, salts of the isopropyl cresol and isopropyl-o-cresol compounds are present in a ratio of between about 1:99 by weight, more preferably between about 3:97 by weight, most preferably about 5:95 by weight. The combination of base reacted antimicrobial compounds appears to have a synergistic effect.

As used herein, the term “synergistic effect” refers to a phenomenon whereby the effect of two or more compounds together is greater then the sum of their effects when used individually. For example, whereas a pharmaceutical composition containing 100 mg of an organic phenolic sodium salt may be needed to treat an infection in an animal, a pharmaceutical composition containing only 45 mg of organic phenolic sodium salt and 45 mg of organic phenolic potassium salt may be needed to treat the same infection in the same animal. An example of the synergistic effect of a combination of antimicrobial compounds is shown in Example 16.

To form the pharmaceutical composition containing a combination of a sodium and potassium organic phenolic salts, a solution containing the desired ratio of antimicrobial compounds is typically mixed at room temperature (e.g., about 20° C. to about 30° C., more typically about 23° C. to about 28° C.) for about 1-10 minutes, preferably about 2-5 minutes at a speed of about 25-100 RPM, preferably 50-75 RPM.

B. Illustrative Pharmaceutical Compositions Containing Antimicrobial Compound for Administration to Humans

A number of different formulations of the antimicrobial compounds of the invention are possible, presented below are some illustrative examples of formulations.

1. Subcutaneous, Intradermal or Intramuscular Injection Formulation

The antimicrobial compounds can be formulated into a parenteral preparation that can be injected subcutaneously, intradermally, or intramuscularly. For such preparations, antimicrobial compound can be present at varying concentrations, for example, between 0.5 wt % to 15 wt %; 3 wt % to 10 wt %; or 5 wt % to 7.5 wt % antimicrobial compound can be combined with a carrier making up the remainder. Preferably, the carrier is nonaqueous, and more preferably it is a purified olive oil.

The antimicrobial compound and the carrier are typically combined in a mixer and mixed at 500 revolutions/minute for 5 minutes. After the formulation is mixed, it is preferably be sterilized, more preferably with U.V. radiation. Once the formulation has been sterilized, it is ready to be injected or packaged for storage.

2. Intravenous Injection Formulation

The antimicrobial compounds can also be formulated into a parenteral preparation that can be injected intravenously. For such preparations, the antimicrobial compound or compounds can be present at varying concentrations, for example between about 0.1 wt % to about 1.0 wt %, more typically between about 0.5% to about 0.8% antimicrobial compounds with a carrier acceptable for parenteral preparations making up the remainder. Preferably, the carrier is sterilized water or an aqueous carrier, and more preferably the carrier contains 0.5 wt % to 1.0 wt % sodium chloride.

The intravenous injectable parenteral preparation is prepared by combining the antimicrobial compound or compounds with the carrier. After the formulation is mixed, it is preferably be sterilized, using known methods. Once the formulation has been sterilized, it is ready to be injected or packaged, preferably in dark bottles or plastic packaging, for storage.

C. Illustrative Dosages for Humans

Dosage amount of the antimicrobial compound(s) will typically depend on the age, weight and diagnosis of the patient, as well as the stage and degree of the affliction.

Sample dosages for treatment of inflammation of the internal organs (lungs, kidneys for example) by intramuscular injection are provided in the table below.

| Patient Type | Dosage Amount per Day | Treatment Time |
|---|---|---|
| Adults | 5.0-7.0 cc | 5 days* |
| Infants 1-12 months | 0.5-1.0 cc | 5 days* |
| Children 1-3 years | 1.0 cc | 5 days* |
| Children 3-7 years | 2.0 cc | 5 days* |
| Youths 7-10 years | 3.0 cc | 5 days* |
| Youths 10-18 years | 4.0 cc | 5 days* |

*If the infection in chronic, the treatment time should be extended to 10 days.

For treatment of sepses (Salmonella spp., E. Coli, Colstridium spp., etc.) by intravenous injection the following dosages can be used.

| Patient Type | Dosage Amount per Day | Treatment Time |
|---|---|---|
| Adults | 500 ml | 7-10 days* |
| Children 1-5 years | 50-100 ml | 5-7 days* |
| Children 5-10 years | 150-250 ml | 5-7 days* |
| Youths 10-20 years | 300-450 ml | 5-7 days* |

*Or until the presence of the pathogens in the blood stream is sufficiently reduced.

III. Infections

The pharmaceutical composition of the invention can be used to treat a variety of internal and external infections in humans and other animals, for example, infections caused by E. coli, Salmonella spp, Pasteurella spp., Staphyloccocus spp., Streptoccocus spp., Corinebacterium spp., Bacillus spp., including Bacillus anthracis, Clostridium spp., Spherophorus spp., Candida spp., Trychophyton spp., Microsporum spp., Micobacterium spp., Vibrio spp., Cryptosporidia spp., Microsporidia spp., Listeria monocytogenes, Lawsonia intracellularis, Treponema desynteriae, Enteroccocus spp., Heamophylus spp., Campylobacter spp., Chlamydia, Brucella spp., and other pathogens, including bacteria, fungi or protozoa.

Examples of illnesses caused by microbial infection that can be treated using the pharmaceutical composition of the invention include internal infections, such as infections of the lungs (for example, pneumonia), kidneys, joints, throat, muscles, and organs, such as the tonsils, sepsis, otitis, sinusitis, conjunctivitis, mastitis, metritis, gastro-enteritis caused by bacteria, fungi, or protozoa, pleuritis, peritonitis, tendonitis, and wounds infected by bacteria. External infections can also be treated, such as dermatitis and boils, also known as abscesses and furuncles, flegmonas and dermatitis.

### WORKING EXAMPLES

### Example 1

### Extraction of Isopropyl-l-cresol and Isopropyl Cresol from Nepeta cataria

Isopropyl-o-cresol and isopropyl-cresol were extracted from Nepeta cataria using a two stage distillation process. In the first stage, dried leaves were extracted using a steam distillation process. After the distillation, the oil is cooled to room temperature for at least 72 hours.

The oil from the steam distillation process was then re-distilled in a second stage distillation process. In the re-distillation, the oil was heated to a temperature of about 186° C. for about 1 hour to remove remaining impurities such as linalool, barneol, pimen, cimen etc. Generally, the impurities have a boiling point of about 150° C. In contrast, both isopropyl-o-cresol and isopropyl-cresol have a boiling point of about 230° C. to 240° C. Thus, a temperature of 180° C. will typically not remove or damage the organic phenolic products.

The oil is again allowed to cool for at least 72 hours to stabilize the oil.

After the oil is cooled, the redistillation is repeated at a temperature of 180° C. for 30 minutes to eliminate almost all of the remaining impurities. The double re-distillation process produced an oil having a purity between 95% and 98%.

After the second re-distillation the oil was allowed to cool for at least 72 hours before production.

### Example 2

### Separation of Isopropyl-o-cresol from Isopropyl-cresol

Isopropyl-o-cresol and isopropyl-cresol were separated by incubating the distilled oil at a temperature of −25° C. for 6 hours. Isopropyl-o-cresol remains as a liquid and isopropyl-cresol is precipitated out as crystals. The two compounds were then separated via filtration.

### Example 3

### Formation of an Antimicrobial Compound with a Group I base

An antimicrobial compound (sodium isopropyl-o-cresol) was formed by combining isopropyl-o-cresol from Example 2 with sodium hydroxide. 55 grams isopropyl-o-cresol was combined with 45 grams of sodium hydroxide (95% purity). The solution was then manually mixed and allowed to react.

### Example 4

### Formation of an Antimicrobial Compound with a Group I base

An antimicrobial compound (potassium isopropyl-o-cresol) was formed by combining isopropyl-o-cresol from Example 2 with potassium hydroxide. 55 grams isopropyl-o-cresol was combined with 45 grams of sodium hydroxide (95% purity). The solution was then manually mixed and allowed to react.

### Example 5

### Formation of an Antimicrobial Compound with a Group I base

An antimicrobial compound (sodium isopropyl cresol) was formed by combining isopropyl cresol from Example 2 with sodium hydroxide. 55 grams isopropyl cresol was combined with 45 grams of sodium hydroxide (95% purity). The solution was then manually mixed and allowed to react.

### Example 6

### Formation of an Antimicrobial Compound with a Group I base

An antimicrobial compound (potassium isopropyl cresol) was formed by combining isopropyl cresol from Example 2 with potassium hydroxide. 55 grams isopropyl cresol was combined with 45 grams of sodium hydroxide (95% purity). The solution was then manually mixed and allowed to react.

### Example 7

### Unmodified Essential Oil

Essential oil was extracted from Origanum vulgaris by steam distillation essentially as described in PCT/NL96/00210. Briefly, the leaves and blossoms of the plants were dried and placed in a distiller. A water source positioned under the leaves and blossoms was heated to about 100° C. under a pressure of about 20 bar for about 2 to about 3 hours. The extracted oil was removed from the distillation column and allowed to cool for at least 72 hours.

### Example 8

### Formulation

A 10% liquid formulation was prepared by combining 50 ml organic phenolic sodium salt (sodium isopropyl cresol) from Example 5 and 50 ml organic phenolic potassium salt (potassium isopropyl cresol) from Example 6. The combination was mixed at room temperature for 5 minutes at a speed of 350 RPM.

For Examples 9 through 32 below, the following terms or phrases, when used, have the following meanings.

Successful recovery means that the animal has improved clinical symptoms. For example, a normalization of body temperature, the animal has begun to eat if not eating before treatment, the specific symptoms (e.g. diarrhea, coughing, etc.) have ceased. In the case of mastitis, a successful recovery is found if the udder began to produce good quality milk and edemas present on the udder disappeared.

An animal was considered healthy if for example, the body temperature was within a normal range, respiration was physiologically correct, symptoms of illness were not present, and the animal had a normal appetite.

Previous treatments if carried out were generally through methods normally used to those in the agricultural field, including oral administration, injection into the animal, or mixed with the feed for example.

Comparative examples are offered for a number of the examples presented below. An example was considered comparative if the animal being treated was the same species, the animal was relatively close to the same age or size, and the condition being treated was diagnosed as the same thing.

### Example 9

### In Vivo Treatment of Pneumonia in Cows

Pneumonia is a common, and frustrating problem in cattle. Pneumonia is basically an inflammation of the tissues of the lungs that results from the response of the animal to an infectious agent. The symptoms of pneumonia include an increased respiratory rate (panting), fever (a rectal temperature of over 102.5° F.), coughing, loss of appetite, and nasal discharge (mucus). The severity of pneumonia can range from mild to rapidly fatal.

The cause of pneumonia, although often attributed to a single syndrome, can have several different causes that include both viral and bacterial agents. Common viruses that can initiate pneumonia include infectious bovine rhinotracheitis virus (IBR), herpes virus, bovine respiratory syncytial virus (BRSV), parainfluenza 3 virus (PI3), certain rhinoviruses, as well as various other viruses. Often, a virus will cause tissue damage, followed by invasion of the compromised tissues by bacteria. The bacteria most often involved in this pattern include Pasteurella hemolytic, Pasteurella multocida, Mycoplasma spp., and Actinomyces spp.

Generally treatment of pneumonia is undertaken using antibiotics. Such treatments are generally ineffective because antibiotics have no effect on viruses, including those that cause pneumonia, and will only kill bacteria if the strain of bacteria present is susceptible to the particular antibiotic being used. Another disadvantage of antibiotics is that they must be given for a period of time, and in a high enough dosage that is great enough to kill the bacteria so that resistance is not developed.

The test population consisted of one milk cow with pneumonia.

Treatment of the cow was accomplished with intramuscular injections of a formulation containing 10% antimicrobial compound and 90% carrier (olive oil).

The antimicrobial compound was formed from extracted essential oils in combination with a salt. The compound included 47.5% sodium isopropyl-o-cresol; 47.5% potassium isopropyl-o-cresol; 2.5% sodium isopropyl-cresol; and 2.5% potassium isopropyl-cresol. The antimicrobial compounds were then combined with neutralized olive oil to form a pharmaceutical composition suitable for intramuscular injection.

The treatment protocol was as follows:

Day 1: 25 ml intramuscular injection morning and night

Day 2: 25 ml intramuscular injection morning and night

The milk cow was cured of the pneumonia within two (2) days.

### Example 10

### In Vivo Treatment of Pneumonia in Calf

One (1) 2½ month old female Holstein calf with an initial weight of 320 lbs (160 kg) was afflicted with acute bronchio pneumonia. Symptoms included coughing, and dripping liquid from the nose. The calf had been positively diagnosed for pneumonia and had been previously treated with ampicillin.

The therapy included three (3) intramuscular injections of 10 ml of the same composition used in Example 9 every 12 hours. The temperature of the calf over the course of treatment is shown below.

| | |
|---|---|
| Initial | 105.8° F. (41° C.) |
| 12 hours | 104.3° F. (40.3° C.) |
| 24 hours | 104° F. (40° C.) |
| 48 hours | 103° F. (39° C.) |
| Final | 103° F. (39° C.) |

The calf made a successful recovery and was healthy with a final weight of 310 lbs (155 kg).

### Example 11

### In Vivo Treatment of Pneumonia in Calf

One 2 month old male Holstein calf with an initial weight of 148 lbs (67 kg) was afflicted with pneumonia. Symptoms included coughing and high fever. The calf had been positively diagnosed for pneumonia and had been previously treated with ampicillin.

The therapy included six (6) intramuscular injections of 10 ml of the composition used in Example 9 every 12 hours. The temperature of the calf over the course of the treatment is shown below.

| | |
|---|---|
| Initial | 106° F. (42° C.) |
| 12 hours | 105° F. (41° C.) |
| 24 hours | 104.8° F. (40.8° C.) |
| 48 hours | 104.3° F. (40.3° C.) |
| Final | 104.1° F. (40.1° C.) |

The calf made a successful recovery.

### Example 12

### In Vivo Treatment of Pneumonia in Calf

One (1) 6 month old Simmental male calf with an initial weight of 573.3 lbs (260 kg) was affected with pneumonia. Symptoms included a fever, and coughing. The horse had not been previously treated.

The therapy included four (4) treatments of intramuscular injection of 20 ml of the composition used in Example 9 every 12 hours. The temperature of the calf over the course of treatment is shown below.

| | |
|---|---|
| Initial | 104.9° F. (40.9° C.) |
| 12 hours | 104° F. (40° C.) |
| 24 hours | 104° F. (40° C.) |
| 48 hours | 103.3° F. (39.3° C.) |
| Final | 103.1° F. (39.1° C.) |

The calf made a successful recovery.

### Example 13a

### In Vivo Treatment of Pneumonia in Cow

One (1) 5 year old female Holstein cow with an initial weight of 1102.5 lbs (500 kg) was affected with pneumonia. Symptoms included fever and coughing. The cow had not been previously treated.

The therapy included four (4) treatments of intramuscular injection of 50 ml of the composition used in Example 9 every 12 hours. The temperature of the cow over the course of treatment is shown below.

| | |
|---|---|
| Initial | 104.5° F. (40.5 ° C.) |
| 12 hours | 103.5° F. (39.5° C.) |
| 24 hours | 103.2° F. (39.2° C.) |
| 48 hours | 103° F. (39° C.) |
| Final | 103° F. (39° C.) |

The cow made a successful recovery.

### Example 13b

### In Vivo Treatment of Pneumonia in Cow

One (1) 15 month old Holstein bull with an initial weight of 990 lbs (450 kg) was affected with pneumonia. Symptoms included fever. The bull had not been previously treated.

The therapy included eight (8) treatments of intramuscular injection of antibiotic every 24 hours. The temperature of the bull over the course of treatment is shown below.

| | |
|---|---|
| Initial | 105.4° F. |
| 12 hours | 105.1° F. |
| 24 hours | 104.5° F. |
| 48 hours | 104.9° F. |
| Final | 103.6° F. |

The bull made a successful recovery.

### Example 13c

### In Vivo Treatment of Pneumonia in Cow

One (1) 8 month old Angus bull with an initial weight of 630 lbs (285 kg) was affected with pneumonia. Symptoms included fever, and coughing. The bull had not been previously treated.

The therapy included seven (7) treatments of intramuscular injection of antibiotic every 24 hours. The temperature of the bull over the course of treatment is shown below.

| | |
|---|---|
| Initial | 104.9° F. |
| 12 hours | 104.4° F. |
| 24 hours | 103.8° F. |
| 48 hours | 103.5° F. |
| Final | normal |

The bull made a successful recovery but lost a significant amount of weight (89 lbs).

### Example 13d

### In Vivo Treatment of Pneumonia in Cows

The test population consisted of five (5) cows, aged 3-5 years with sub-chronic infections. The cows were experiencing fever (41.5° C.), a reduced milk production, and were having no reaction to the antibiotic treatment being administered.

A formulation containing 7.5% antimicrobial compound and 92.5% carrier (olive oil) was prepared. The antimicrobial compound included 47.5% sodium isopropyl-o-cresol; 47.5% potassium isopropyl-o-cresol; 2.5% sodium isopropyl-cresol; and 2.5% potassium isopropyl-cresol. The treatment protocol was as follows:

Day 1: 50 ml intramuscular injection at morning and night.

Day 2: 40 ml intramuscular injection at morning and 50 ml intramuscular injection at night.

Days 3-5: 30 ml intramuscular injection at morning and 30 ml intramuscular injection at night

The results from the treatment protocol above are as follows. 24 hours after treatment began, the fever present in the afflicted cows returned to normal (39° C.). 48 hours after the treatment began, respiratory difficulties in the afflicted cows disappeared. 72 hours after treatment began, milk production began to rise back to a normal level. After 5 days of treatment, all afflicted cows were free from all symptoms.

### Example 14a

### In Vivo Treatment of Mastitis in Cow

One (1) 5 year old Holstein cow with an initial weight of 1000 lbs. (500 kg) with sub-chronic mastitis was treated. Symptoms included inflammation of the udder. The cow had previously been treated with different antibiotics.

The therapy included three (3) intramuscular injections of 50 ml of the composition used in Example 9 every 12 hours. The temperature of the cow over the course of treatment is shown below.

| | |
|---|---|
| Initial | 104.3° F. (40.3° C.) |
| 12 hours | 104° F. (40° C.) |
| 24 hours | 103.5° F. (39.5° C.) |
| 48 hours | 103.1° F. (39.1° C.) |
| Final | 103.1° F. (39.1° C.) |

The cow made a successful recovery and was healthy.

### Example 14b

### Comparative in Vivo Treatment of Mastitis in Cow

One (1) 4 year old female Holstein cow with an initial weight of 1210 lbs. (550 kg) with mastitis was treated. Symptoms included edema of the udder, blood in the milk, and a high temperature. The cow had not been previously treated.

The therapy included six (6) intramuscular injections of antibiotic every 24 hours. The temperature of the cow over the course of treatment is shown below.

| | |
|---|---|
| Initial | 105.6° F. |
| 12 hours | 105.3° F. |
| 24 hours | 104.3° F. |
| 48 hours | 104.4° F. |
| Final | 103.5° F. |

The cow made a successful recovery and was healthy.

### Example 14c

### In Vivo Treatment of Mastitis in Cow

One (1) 6 year old Holstein cow with an initial weight of 1210 lbs. (500 kg) with mastitis was treated. Symptoms included edema in the udder, and blood in the milk. The cow had not been previously treated with different antibiotics.

The therapy included fourteen (14) applications of antibiotic to the udder every 12 hours. The temperature of the cow over the course of treatment is shown below.

| | |
|---|---|
| Initial | 106.3° F. |
| 12 hours | 105.6° F. |
| 24 hours | 104.9° F. |
| 48 hours | 104.3° F. |
| Final | 102.7° F. |

The udder of the cow was unable to be used after treatment. The animal was eventually brought to slaughter because it was no longer useful.

### Example 15

### In Vivo Treatment of Mastitis in Cow

One (1) 5 year old Holstein cow with an initial weight of 1213 lbs (550 kg) was afflicted with mastitis. Symptoms included edema of the udder and poor quality milk. The cow had not been previously diagnosed or treated.

The therapy included three (3) intramuscular injections of 50 ml of the composition used in Example 9 every 12 hours. The temperature of the cow over the course of treatment is shown below.

| | |
|---|---|
| Initial | 104.7° F. (40.7° C.) |
| 12 hours | 104.2° F. (40.2° C.) |
| 24 hours | 104° F. (40° C.) |
| 48 hours | 103.5° F. (39.5° C.) |
| Final | 103° F. (39° C.) |

The cow made a successful recovery.

### Example 16a

### In Vivo Treatment of Diarrhea in Calf

One (1) 12 day old female Simmental calf with an initial weight of 132.3 lbs (60 kg) was afflicted with cryptosporidiosis. The calf had profuse diarrhea and a high fever. The calf had been previously treated with different antibiotics. Microbiological testing before the treatment was positive for cryptsporidiosis.

The therapy included six (6) intramuscular injections of 10 ml of the composition used in Example 9 every 12 hours. The temperature of the calf over the course of treatment is shown below.

| | |
|---|---|
| Initial | 105.3° F. (41.3° C.) |
| 12 hours | 104.8° F. (40.8° C.) |
| 24 hours | 104.5° F. (40.5° C.) |
| 48 hours | 104° F. (40° C.) |
| Final | 103.8° F. (39.8° C.) |

The calf made a successful recovery.

### Example 16b

### In Vivo Treatment of Diarrhea in Calf

One (1) 10 day old male Angus calf with an initial weight of 114.4 lbs (52 kg) was afflicted with diarrhea. The calf had profuse diarrhea, a fever, and was dehydrated. The calf had not been previously treated. Microbiological testing before the treatment was positive for E. coli.

The therapy included eight (8) intramuscular injections of antibiotics every 12 hours. The temperature of the calf over the course of treatment is shown below.

| | |
|---|---|
| Initial | 105.8° F. |
| 12 hours | 105.8° F. |
| 24 hours | 106.2° F. |
| 48 hours | 105.8° F. |
| Final | death |

The calf did not recover, and died.

### Example 17

### In Vivo Treatment of Kidney Inflammation in Calves

The test population consisted of 2 calves, age 30 days, with an acute form of kidney inflammation. The calves were experiencing a slight fever of 39° C., and had blood in the urine.

A formulation containing 5.0% antimicrobial compound and 95% carrier (olive oil) was prepared. The antimicrobial compound included 47.5% sodium isopropyl-o-cresol; 47.5% potassium isopropyl-o-cresol; 2.5% sodium isopropyl-cresol; and 2.5% potassium isopropyl-cresol. The treatment protocol was as follows:

Day 1: 15 ml intramuscular injection.

Day 2: 10 ml intramuscular injection

Day 3: 10 ml intramuscular injection

The results from the treatment protocol above are as follows. 24 hours after treatment began, the amount of blood in the urine had decreased. 48 hours after treatment, the blood in the urine had disappeared. 72 hours after treatment began, all both calves were completely recovered.

### Example 18

### In Vivo Treatment of Pneumonia in Hogs

Pneumonia can be similarly difficult to deal with in pig populations. Symptoms are generally the same as seen in cattle, increased respiratory rate (panting), fever (a rectal temperature of over 102.5° F.), coughing, loss of appetite, and nasal discharge (mucus). Pneumonia affects pigs of all ages, beginning with those as young as 7-10 days. It is estimated that 85% or more of the swine herds in the Midwestern United States are infected with pneumonia.

There are three recognized species of Mycoplasma bacteria that are generally thought to cause pneumonia in pigs, Mycoplasma hyopneumoniae, Mycoplasma hyorhisnis, and Mycoplasma hyosynoviae.

The first test population consisted of one 100 pound guilt with acute pneumonia.

Treatment of the pig was accomplished with intramuscular injections of antimicrobial compounds of Example 9. The treatment protocol was as follows:

Day 1: 15 ml intramuscular injection morning and night

Day 2: 15 ml intramuscular injection morning and night

The guilt was cured of the pneumonia after the third injection of the antimicrobial

The next test population consisted of one 250 lb guilt with pneumonia. The hog was suffering from rapid breathing, wheezing and excessive mucus. The treatment was conducted with the same formulation with the following protocol.

Day 1: 10 ml intramuscular injection morning and night

Day 2: 10 ml intramuscular injection morning and night

The guilt was cured of the pneumonia within two days.

### Example 19

### In Vivo Treatment of Pneumonia in Hogs

The test population consisted of 35 hogs, age 70 days, with acute pneumonia. The hogs were experiencing difficulty in respiration and a fever of 42° C. There had been no antibiotic treatment because it was an organic farm that did not allow the use of antibiotics.

The treatment protocol was as follows with the same formulation used in Example 17.

Day 1: 10 ml intramuscular injection.

Day 2: 5 ml intramuscular injection

Day 3: 5 ml intramuscular injection

The results from the treatment protocol above are as follows. 24 hours after treatment began, the temperature of the hogs returned to normal (39.5° C.). 48 hours after treatment, none of the hogs previously afflicted with respiratory difficulties were experiencing such symptoms. 72 hours after treatment began, all hogs treated were recovered.

### Example 20

### In Vivo Treatment of Pneumonia in Pig

One (1) 3.5 year old Landras sow with an initial weight of 529.2 lbs (240 kg) afflicted with bronchial pneumonia. Symptoms included high temperature. The sow was positively diagnosed for pneumonia and had been previously treated with penicillin.

The therapy included three (4) intramuscular injections of 15 ml of the composition used in Example 9 every 12 hours. The temperature of the sow over the course of treatment is shown below.

| | |
|---|---|
| Initial | 105.3° F. (41.3° C.) |
| 12 hours | 104.3° F. (40.3° C.) |
| 24 hours | 104° F. (40° C.) |
| 48 hours | 103° F. (39° C.) |
| Final | 102.8° F. (38.8° C.) |

The sow made a successful recovery.

### Example 21

### In Vivo Treatment of Metritis, Mastitis, Agaloctie in Pig

One (1) 3 year old Landras sow with an initial weight of 450 lbs. (220 kg) afflicted with metritis, mastitis and agaloctie. Symptoms included elevated temperature and excretion from the uterus. Her piglets had diarrhea. No previous treatment or diagnosis.

The therapy included two intramuscular injections of 20 ml of the composition used in Example 9 every 12 hours. The temperature of the sow over the course of the treatment is shown below.

| | |
|---|---|
| Initial | 104° F. (40° C.) |
| 12 hours | 103° F. (39° C.) |
| 24 hours | 102.5° F. (38.5° C.) |
| 48 hours | 102° F. (38° C.) |
| Final | 102° F. (38° C.) |

The sow made a successful recovery and was healthy with a final weight of 450 lbs. (220 kg).

### Example 22

### In Vivo Treatment of Mastitis, Metritis, Agalactica (MMA) in Sow

One (1) 18 month old Yorkshire, Rijetrew female sow with an initial weight of 396.9 lb (180 kg) was affected with MMA. Symptoms included excretion from the uterus, edema of the udder, and a lack of milk. The sow had not been previously treated.

The therapy included four (4) treatments of intramuscular injection of 20 ml of the composition used in Example 9 every 12 hours. The temperature of the sow over the course of treatment is shown below.

| | |
|---|---|
| Initial | 105.3° F. (41.3° C.) |
| 12 hours | 104.4° F. (40.4° C.) |
| 24 hours | 103.9° F. (39.9° C.) |
| 48 hours | 103.2° F. (39.2° C.) |
| Final | 103.1° F. (39.1° C.) |

The sow made a successful recovery.

### Example 23a

### In Vivo Treatment of Diarrhea in Piglet

One (1) 25 day old male Landras piglet with an initial weight of 20 lbs. (10 kg) afflicted with E. coli diarrhea. The piglet had been positively diagnosed for E. coli and had not been previously treated.

The therapy included three (3) intramuscular injections of 3 ml of the composition used in Example 9 every 12 hours. The temperature of the piglet over the course of treatment is shown below.

| | |
|---|---|
| Initial | 105° F. (41° C.) |
| 12 hours | 104° F. (40° C.) |
| 24 hours | 104.5° F. (40.5° C.) |
| 48 hours | 104° F. (40° C.) |
| Final | 103.5° F. (39.5° C.) |

The piglet made a successful recovery.

### Example 23b

### In Vivo Treatment of Diarrhea in Piglet

One (1) 28 day old female Landras piglet with an initial weight of 15.4 lbs. (7 kg) afflicted with E. coli diarrhea. The piglet had been positively diagnosed for E. coli and had not been previously treated.

The therapy included three (3) intramuscular injections of antibiotic every 12 hours. The temperature of the piglet over the course of treatment is shown below.

| | |
|---|---|
| Initial | 106.3° F. |
| 12 hours | 106.3° F. |
| 24 hours | 106.2° F. |
| 48 hours | 106.5° F. |
| Final | death |

The piglet died.

### Example 24

### In Vivo Treatment of Diarrhea in Pig

One (1) 3 month old male Yorkshire pig with an initial weight of 88 lbs (40 kg) was afflicted with profuse bloody diarrhea. The pig had not been previously diagnosed or treated.

The therapy included six (6) intramuscular injections of 10 ml of the composition used in Example 9 every 12 hours. The temperature of the pig over the course of treatment is shown below.

| | |
|---|---|
| Initial | 103.8° F. (39.8° C.) |
| 12 hours | 103.5° F. (39.5° C.) |
| 24 hours | 103.4° F. (39.4° C.) |
| 48 hours | 103.4° F. (39.4° C.) |
| Final | 103.4° F. (39.4° C.) |

The pig made a successful recovery.

### Example 25

### In Vivo Treatment of Diarrhea in Pig

One (1) 30 day old male Yorkshire pig with an initial weight of 22.05 lbs (10 kg) was afflicted with diarrhea. The symptoms included profuse diarrhea and dehydration. The pig had not been previously diagnosed or treated. Microbiological testing before the treatment began was positive for E. coli.

The therapy included four (4) intramuscular injections of 3 ml of the composition used in Example 9 every 12 hours. The temperature of the pig over the course of treatment is shown below.

| Initial | | 105° F. (41° C.) |
|---|---|---|
| 12 | hours | 104.2° F. (40.2° C.) |
| 24 | hours | 103.9° F. (39.9° C.) |
| 48 | hours | 103.7° F. (39.7° C.) |

| | |
|---|---|
| Final | 103.5° F. (39.5° C.) |

The pig made a successful recovery.

### Example 26

### In Vivo Treatment of Glomerulonephritis in Horse

One (1) eight year old Englander female horse with an initial weight of 1102 lbs. (500 kg) was afflicted with glomerulonephritis. Symptoms included frequent urination and blood in urine. The horse had been tested for E. coli and treated with different antibiotics.

The therapy included ten (10) intramuscular injections of 25 ml of the composition used in Example 9 every 12 hours. The temperature of the horse over the course of treatment is shown below.

| Initial | | 103° F. (39° C.) |
|---|---|---|
| 12 | hours | 102.8° F. (38.8° C.) |
| 24 | hours | 102.5° F. (38.5° C.) |
| 48 | hours | 102.4° F. (38.4° C.) |

| | |
|---|---|
| Final | 101.8° F. (37.8° C.) |

The horse made a successful recovery and was healthy.

### Example 27

### In Vivo Treatment of Tendon Inflammation in Horse

One (1) 6 year old Lipicaner male horse with an initial weight of 1036 lbs (470 kg) was afflicted with inflammation of tendons. Symptoms included difficulty walking, edema on the lower leg. The horse had not been previously diagnosed or treated.

The therapy included six (6) intramuscular injections of 30 ml of the composition used in Example 9 every 12 hours. The temperature of the horse over the course of treatment is shown below.

| Initial | | 102.3° F. 38.3° C.) |
|---|---|---|
| 12 | hours | 102.2° F. (38.2° C.) |
| 24 | hours | 102° F. (38° C.) |
| 48 | hours | 102° F. (38° C.) |

| | |
|---|---|
| Final | 102° F. (38° C.) |

The horse made a successful recovery.

### Example 28

### In Vivo Treatment of Parastitis in Horse

One (1) 11 year old female horse with an initial weight of 1168.6 lbs (530 kg) was affected with parastitis, inflammation of the saliva gland. Symptoms included edema of the saliva gland. The horse had previously been treated with antibiotics.

The therapy included two (2) treatments of intramuscular injection of 50 ml of the composition used in Example 9 every 12 hours. The temperature of the horse over the course of treatment is shown below.

| Initial | | 104° F. (40° C.) |
|---|---|---|
| 12 | hours | 103.1° F. (39.1° C.) |
| 24 | hours | 102.5° F. (38.5° C.) |
| 48 | hours | 102.1° F. (38.1° C.) |

| | |
|---|---|
| Final | 102° F. (38° C.) |

The horse made a successful recovery.

### Example 29

### In Vivo Treatment of Pneumonia in Dog

One (1) 6 year old female Saint Bernard with an initial weight of 132.3 lbs (60kg) was affected with pneumonia. Symptoms included a high fever and coughing. The dog had been previously treated with various antibiotics.

The therapy included six (6) treatments of intramuscular injection of 10 ml of the composition used in Example 9 every 12 hours. The temperature of the dog over the course of treatment is shown below.

| Initial | | 105.1° F. (41.1° C.) |
|---|---|---|
| 12 | hours | 104.3° F. (40.3° C.) |
| 24 | hours | 103.2° F. (39.2° C.) |
| 48 | hours | 102.4° F. (38.4° C.) |

| | |
|---|---|
| Final | 102.1° F. (38.1° C.) |

The dog made a successful recovery.

### Example 30

### In Vivo Treatment of Pneumonia in Dog

One (1) 7 year old male Labrador retriever with an initial weight of 55 lbs (25 kg) was affected with pneumonia. Symptoms included a high fever. The dog had not been previously treated.

The therapy included four (4) treatments of intramuscular injection of 5 ml of the composition used in Example 9 every 12 hours. The temperature of the dog over the course of treatment is shown below.

| Initial | | 104.9° F. (40.9° C.) |
|---|---|---|
| 12 | hours | 104.1° F. (40.1° C.) |
| 24 | hours | 103.4° F. (39.4° C.) |
| 48 | hours | 102.3° F. (38.3° C.) |

| | |
|---|---|
| Final | 102° F. (38° C.) |

The dog made a successful recovery.

### Example 31

### In Vivo Treatment of Nephritis in Dog

One (1) 5 year old male poodle with an initial weight of 33 lbs (15 kg) was affected with nephritis, kidney inflammation. Symptoms included frequent, difficult urination. The dog had been previously treated with various antibiotics. Microbiological testing before treatment was positive for E. coli.

The therapy included eight (8) treatments of intramuscular injection of 5 ml of the composition used in Example 9 every 12 hours. The temperature of the dog over the course of treatment is shown below.

| Initial | | 104.3° F. (40.3° C.) |
|---|---|---|
| 12 | hours | 103.4° F. (39.4° C.) |
| 24 | hours | 103° F. (39° C.) |
| 48 | hours | 102.4° F. (38.4° C.) |

| | |
|---|---|
| Final | 102.2° F. (38.4° C.) |

The dog made a successful recovery.

### Example 32

### In Vivo Treatment of Arthritis of Dog

One (1)11 year old male English Terrier with an initial weight of 22.05 lbs (10 kg) was affected with arthritis. Symptoms included edema and joint inflammation. The dog had been previously treated with cortisone and different antibiotics.

The therapy included six (6) treatments of intramuscular injection of 5 ml of the composition used in Example 9 every 24 hours. The temperature of the dog over the course of treatment is shown below.

| Initial | | 102.3° F. (38.3° C.) |
|---|---|---|
| 12 | hours | 102.3° F. (38.3° C.) |
| 24 | hours | 102.2° F. (38.2° C.) |
| 48 | hours | 102.4° F. (38.4° C.) |

| | |
|---|---|
| Final | 102.2° F. (38.2° C.) |

The dog had improved health after the course of treatment. The treatment was deemed to be partly successful, because the case was considered a chronic case of joint inflammation.

### Example 33

### In vitro MIC Results for Antimicrobial Compound

Minimal inhibitory concentration (MIC) is a standard test for determining levels of microbial resistance to an antibiotic or other compound. The test is generally accomplished by making serial dilutions of the compound in a liquid medium, which has been inoculated with a standardized number of organisms and incubated for a prescribed time. The lowest concentration (highest dilution) of the compound that prevents appearance of turbidity is considered to be the minimal inhibitory concentration (MIC). The results below are MIC results for the composition used in Example 9. They were accomplished using a standard protocol with Mueller Hinton Broth.

CFU/ml

| Bacteria Strain | Positive Control | 0.1% cmpd. | 0.2% cmpd. | 0.3% cmpd. | 0.4% cmpd. |
|---|---|---|---|---|---|
| E. coli K99 | 3.2 × 10⁷ | +* | 0 | 0 | 0 |
| E. coli 987P | 3.6 × 10⁷ | + | 10³ | 0 | 0 |
| E. coli K88 | >10⁷ | + | 0 | 0 | 0 |
| E. coli F18 | 6.0 × 10⁷ | + | 0 | 0 | 0 |
| E. coli 0157:H7 | >10⁷ | + | 0 | 0 | 0 |
| Salmonella | >10⁷ | + | 50 | 0 | 0 |
| typhimurium | | | | | |
| Salmonella | >10⁷ | + | 0 | 0 | 0 |
| cholerasuis | | | | | |
| Pasteurella | 1.2 × 10⁸ | + | 0 | 0 | 0 |
| multocida A | | | | | |
| Pasteurella | 4.0 × 10⁷ | + | 0 | 0 | 0 |
| multocida D | | | | | |
| Strep. suis 1 | 1.0 × 10⁷ | + | 0 | 0 | 0 |
| Strep. suis 3 | 5.0 × 10⁷ | + | 0 | 0 | 0 |
| Strep. suis 5 | 5.0 × 10⁷ | + | 0 | 0 | 0 |
| Strep. suis 7 | 1.0 × 10⁷ | + | 40 | 0 | 0 |

*+ denotes obvious growth in broth

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A pharmaceutical composition for treating an infection in an animal, said composition comprising:
(a) antimicrobial compound; and
(b) a pharmaceutically acceptable carrier for parenteral administration.

2. The pharmaceutical composition of claim 1 wherein the antimicrobial compound comprises an organic phenolic compound.

3. The pharmaceutical composition of claim 2 wherein the organic phenolic compound is reacted with a Group I salt.

4. The pharmaceutical composition of claim 2 wherein the organic phenolic compound is selected from isopropyl-o-cresol, isopropyl-cresol and combinations thereof.

5. The pharmaceutical composition of claim 3 wherein the Group I base is a Group I hydroxide base.

6. The pharmaceutical composition of claim 5 wherein the Group I base is selected from sodium hydroxide, potassium hydroxide and combinations thereof.

7. The pharmaceutical composition of claim 1 wherein the antimicrobial compound comprises isopropyl-o-cresol and isopropyl-cresol chemically reacted with sodium hydroxide and potassium hydroxide.

8. The pharmaceutical composition of claim 1 comprising from 0.1 wt % to 15 wt % antimicrobial compound

9. The pharmaceutical composition of claim 1 comprising from 0.1 wt % to 15 wt % antimicrobial compound, wherein the pharmaceutically acceptable carrier is suitable for subcutaneous, intradermal, or intramuscular administration.

10. The pharmaceutical composition of claim 1 comprising from 3.5% to 10% antimicrobial compound, wherein the pharmaceutically acceptable carrier comprises a vegetable oil.

11. The pharmaceutical composition of claim 10 wherein the pharmaceutically acceptable carrier comprises olive oil.

12. The pharmaceutical composition of claim 1 comprising from 0.1 wt % to 1.0 wt % antimicrobial compound, wherein the pharmaceutically acceptable carrier is suitable for intravenous administration.

13. The pharmaceutical composition of claim 12 comprising from 0.5% to 0.8% antimicrobial compound, wherein the pharmaceutically acceptable carrier comprises 0.5wt % to 1.0 wt % sodium chloride.

14. The pharmaceutical composition of claim 4 wherein the antimicrobial compound comprises more isopropyl-o-cresol than isopropyl cresol.

15. The pharmaceutical composition of claim 1 wherein the antimicrobial compound comprises between 55 wt % and 99 wt % isopropyl-o-cresol or base reacted isopropyl-o-cresol and between 1 wt % and 45 wt % isopropyl cresol or base reacted isopropyl cresol.

16. The pharmaceutical composition of claim 1 wherein the antimicrobial compound comprises between 75 wt % and 99 wt % isopropyl-o-cresol or base reacted isopropyl-o-cresol and between 1 wt % and 25 wt % isopropyl cresol or base reacted isopropyl cresol.

17. The pharmaceutical composition of claim 1 wherein the antimicrobial compound comprises between 90 wt % and 99 wt % isopropyl-o-cresol or base reacted isopropyl-o-cresol and between 1 wt % and 10 wt % isopropyl cresol or base reacted isopropyl cresol.

18. The pharmaceutical composition of claim 1 wherein the antimicrobial compound comprises between 95 wt % and 99 wt % isopropyl-o-cresol or base reacted isopropyl-o-cresol and between 1 wt % and 5 wt % isopropyl cresol or base reacted isopropyl cresol.

19. The pharmaceutical composition of claim 1 wherein the animal is selected from humans, horses, cows, pigs, sheep, goats, rabbits, dogs, cats, chickens, turkeys, ducks and birds.

20. The pharmaceutical composition of claim 1 wherein the infection comprises infection by E. coli, Salmonella spp, Pasteurella spp., Staphyloccocus spp., Streptoccocus spp., Corinebacterium spp., Bacillus spp., Clostridium spp., Spherophorus spp., Candida spp., Trychophyton spp., Microsporum spp., Micobacterium spp., Cryptosporidia spp., Microsporidia spp., Listeria monocytogenes, Lawsonia intracellularis, Treponema desynteriae, Enteroccocus spp., Heamophylus spp., Campylobacter spp., Chlamydia, Brucella spp., or Vibrio spp.

21. A method for treating infection in an animal, said method comprising:
administering to the animal a pharmaceutical composition comprising antimicrobial compound and a pharmaceutically acceptable carrier for parenteral administration.

22. The method of claim 21, wherein said antimicrobial compound comprises an organic phenolic compound.

23. The method of claim 22, wherein the organic phenolic compound is reacted with a Group I base.

24. The method of claim 23, wherein the organic phenolic compound is selected from isopropyl-o-cresol, isopropyl-cresol and combinations thereof.

25. The method of claim 23 wherein the Group I base is a Group I hydroxide base.

26. The method of claim 25 wherein the Group I base is selected from sodium hydroxide, potassium hydroxide and combinations thereof.

27. The method of claim 21 wherein the antimicrobial compound comprises isopropyl-o-cresol and isopropyl-cresol chemically reacted with sodium hydroxide and potassium hydroxide.

28. The method of claim 21 wherein the animal is selected from the group consisting of humans, horses, cows, pigs, sheep, goats, rabbits, dogs, cats, chickens, turkeys, ducks and birds.

29. The method of claim 21 wherein the infection comprises infection by E. coli, Salmonella spp, Pasteurella spp., Staphyloccocus spp., Streptoccocus spp., Corinebacterium spp., Bacillus spp., Clostridium spp., Spherophorus spp., Candida spp., Trychophyton spp., Microsporum spp., Micobacterium spp., or Vibrio spp.

30. The method of claim 21 wherein the infection comprises an internal infection.

31. The method of claim 30, comprising an infection of lungs, kidneys, joints, throat, muscles, or organs.

32. The method of claim 31, comprising an infection of tonsils.

33. The method of claim 21 wherein the infection comprises an external infection.

34. The method of claim 33 wherein the external infection comprises dermatitis or boils.

35. The method of claim 21, wherein said animal is a human animal.

36. Specific independent claim for VBG in composition.
